(19) United States
(12) Patent Application Publication
(10) Pub. No.: US 2003/0091540 A1
(43) Pub. Date: May 15, 2003

(54) Compositions and methods for delivering antibacterial, antifungal and antiviral ointments to the oral, nasal or vaginal cavity

(76) Inventors: Nawaz Ahmad, Monmouth Junction, NJ (US); Shun Lin, Plainsboro, NJ (US); Kalpana J. Patel, West Windsor, NJ (US); Mark Huseth, Somerset, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/978,810

(22) Filed: Oct. 16, 2001

(51) Int. Cl. A01N 63/00; A61K 9/00

(52) U.S. Cl. 424/93.3; 424/400

(57) ABSTRACT

The ointments and methods of the invention treat oral and vaginal fungal and yeast infections. Ointments comprise antifungals and/or antibacterials, and a mixture of water-soluble, and water-insoluble components, which effectively treat oral and/or vaginal infections in a single dose or multiple doses comprise retaining antifungals and/or antibacterials for an effective time above a minimum concentration. Methods of treating the infections in a single dose comprise methods for determining whether ointments will be effective in a single dose test blood for concentration of antifungals and antibacterials at set time intervals.

### FIELD OF THE INVENTION

This invention relates to unique oral and vaginal antifungal ointments and antibacterial ointments intended for a single-dose, or multiple dose application and methods for delivering antifungal ointments and antibacterial ointments to the oral or vagina cavity and determining the residence time of these ointments in the cavity.

Infections in the vagina may be caused by yeast (which is a fungus), called Candida and/or bacteria, most commonly bacterial vaginosis. If these infections are not treated properly, the infections can be very uncomfortable and even painful. Conventionally, these infections are treated locally by creams, suppositories, soft gelatin capsules, vaginal tablets and ointments, which contain antifungals or antibacterials. Treatments can last from seven days to one day.

Fungal and bacterial infections may also occur in the oral cavity. Treatment is difficult because the treating composition must be retained in the oral cavity for a sufficient time for treatment.

### BACKGROUND OF THE INVENTION

Antifungal or antibacterial creams, suppositories and tablets, in the prior art, are available with treatment regimens lasting for seven days or three days, with reapplication required every day. The repeated dosing is very inconvenient and often messy for consumers. There is a consumer preference for one day or single dose application treatments. However, the problem in the prior art has been retaining sufficient antifungal or antibacterial at the infection site for sufficient time to be effective.

Additionally, the existing methods, which are used to characterize the effectiveness of a single-dose vaginal ointment, are expensive, time-consuming clinical efficacy studies. There exists a need for a quicker, cheaper method of comparing antifungal and antibacterial efficacies for single dose applications to the vagina.

### SUMMARY OF THE INVENTION

This invention relates to antifungal and antibacterial ointments for multiple or single application (preferably in seven doses, three day doses or most preferably single doses) to the oral cavity or vagina (also referred to as “vaginal cavity”). The problems seen in the antifungal and antibacterial treatments in the prior art are numerous. Many treatments require multi day treatment and multiple applications per day.

Since the ointments may leak from the vagina or the antifungal or antibacterial agents may leach out of the ointment, a reapplication of the ointment is required. Reapplication is required to insure and maintain a certain minimum concentration of antifungal or antibacterial at the site of infection, and thus is very inconvenient for the consumer.

Treatments applied to the mouth have additional problems. Compositions which are applied to the oral cavity must be retained for a sufficient time in order to deliver an effective amount of antifungal or antibacterial to the infection site. Additionally, the composition must be tolerable to taste, so that the subject does not dilute or remove the composition by rinsing his mouth before the antifungal or antibacterial can be delivered and retained at the infection site for sufficient time.

In accordance with the present invention, a method for treating vaginal fungal and vaginal bacterial infections includes inserting in the vaginal cavity of a mammalian species, including humans, a therapeutic amount of the antifungal or antibacterial ointment and allowing the ointment to melt in the vaginal cavity and adhere to the vaginal membrane.

Additionally, the present invention includes a method for treating oral fungal and oral bacterial infections by inserting in the oral cavity of an animal, including humans, a therapeutic amount of the antifungal or antibacterial ointment and allowing the ointment to melt in the oral cavity and adhere to the mucosal membrane.

An embodiment of the invention comprises an ointment comprising one or more antifungals, one or more water insoluble components and one or more water soluble components. The prior art does not teach the combined use of both water soluble and water insoluble components as a base for an antifungal ointment. Preferably in this invention, the melting point of the ointment is such that a significant part of the ointment melts in response to body heat, thereby facilitating uniform spreading of the ointment. The antifungal is preferably an immidazole derivative, more preferably miconazole nitrate, clotrimazole, econazole, saperconazole, terconazole, fenticonazole, sertaconazole, posaconazole, itraconazole, ketoconazole, butaconazole, tioconazole, fluconazole, cyclopirox, their pharmaceutically acceptable salts, or a combination thereof, and most preferably miconazole nitrate.

In order to promote retention of the ointment in the oral cavity for a sufficient time, the ointment should be tolerable to the animal's sense of taste. In one embodiment of the invention, the ointment may also include natural flavorings, artificial flavorings and mixtures thereof.

An embodiment of the invention comprises an ointment comprising one or more antibacterials, one or more water insoluble components and one or more water soluble components. The antibacterial is preferably metronidazole, secnidazole, ornidazole, tinidazole, clindamycin, sodium polystyrene sulfate, and sodium cellulose sulfate, and most preferably metronidazole.

Another embodiment of the invention comprises an ointment for vaginal use comprising one or more antifungals, one or more water insoluble components, one or more water soluble components, and one or more probiotics. The probiotic is preferably probiotic organism, including but not limited to Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis or B. longum.

Another embodiment of the invention comprises an ointment for vaginal use comprising one or more antibacterial, one or more water insoluble components, one or more water soluble components, and one or more probiotics. The probiotic is preferably probiotic organism, including but not limited to Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis or B. longum.

Another embodiment of the invention comprises an ointment for vaginal use comprising one or more antivirals, one or more water insoluble components, one or more water soluble components, and one or more probiotics. The probiotic is preferably probiotic organism, including but not limited to Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis or B. longum.

Another embodiment of the invention is the method of treating a fungal infection of a body cavity. The body cavity includes, but is not limited to the nose, oral cavity or mouth, and the vagina. An ointment, which comprises an antifungal, and a combination of water soluble and water insoluble components, is applied to the body cavity, preferably only once, and is retained in the body cavity. At least part, and preferably all of, the ointment is melted, preferably on contact with the body and most preferably from body heat. The ointment is spread substantially uniformly in the body cavity. The ointment may comprise an antibacterial in addition to an antifungal.

Another embodiment of the invention comprises an ointment comprising one or more antifungals, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Another embodiment of the invention comprises an ointment comprising one or more antivirals, one or more water insoluble components, and one or more water soluble components. The antiviral may preferably include but is not limited to immunomodulators, more preferably imiquimod, its derivatives, podofilox, podophyllin, interferon alpha, reticulos, and cidofovir.

Another embodiment of the invention comprises an ointment comprising one or more antibacterials, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Another embodiment of the invention is the method of treating a bacterial infection of a body cavity. The body cavity includes, but is not limited to the nose, oral cavity or mouth, and the vagina. An ointment, which comprises an antibacterial, a combination of water soluble and water insoluble components, a bioadhesive agent, and a dispersing agent, is applied to the body cavity, preferably only once, and is retained in the body cavity. At least part, and preferably all of, the ointment is melted, preferably on contact with the body and most preferably from body heat. The ointment is spread substantially uniformly in the body cavity.

The invention also includes a method of treating a viral infection of a body cavity. The body cavity includes, but is not limited to the nose, oral cavity or mouth, and the vagina. An ointment, which comprises an antiviral, and a combination of water insoluble and water soluble components is applied to the body cavity, preferably only once, and is retained in the body cavity. At least part, and preferably all of the ointment is melted, preferably on contact with the body and most preferably from body heat. The ointment is spread substantially uniformly in the body cavity.

The invention also includes a method of identifying a vaginal antifungal ointment, which is effective after a single dose. An antifungal ointment is applied to the vagina of a consumer, and blood samples are taken at set time interval, preferably at 2, 4, 8, 12, 16, 24, 48,72, 96, 120 and 144 hours. The samples are tested for the concentration of antifungal in the blood, and the data is recorded. Finally, it must be determined whether the data is above a minimum concentration for antifungal, preferably 1.0 g/ml and below a maximum concentration below its toxicity level for at least as long a time to be effective for treating the infection, preferably over about 24 hours, most preferably for about 72 to about 120 hours. This method may be cheaper and more time-efficient than the prior art clinical testing of vaginal antifungal ointments.

Another embodiment of the invention includes any combination of the methods of treating a bacterial infection, a fungal infection and a viral infection.

The invention also includes a method of identifying a vaginal antibacterial ointment, which is effective after a single dose. An antibacterial ointment is applied to the vagina of a consumer, and blood samples are taken at set time intervals. The samples are tested for the concentration of antibacterial in the blood, and the data is recorded. Finally, it must be determined whether the data is above a minimum concentration of antibacterial, and below a maximum concentration, for at least as long a time to be effective for treating the infection, preferably over about 24 hours, most preferably from about 72 to about 120 hours. This method may be cheaper and more time-efficient than the prior art clinical testing of vaginal antibacterial ointments.

### BRIEF DESCRIPTION OF THE DRAWINGS

The invention will become more readily apparent from the following description of the accompanying drawings wherein:

FIG. 1 is a graph showing the absorption profile of an ideal single-dose vaginal antifungal ointment.

FIG. 2 is a graph showing the comparison of absorption profiles of the prior art.

FIG. 3 is a graph showing the comparison of absorption profiles of several embodiments of the invention and some prior art compositions.

### DETAILED DESCRIPTION OF THE PREFERRED EMBODIMENTS AND DRAWINGS

The present invention is not to be limited by any mechanism described in the specification, because it is defined by the claims.

An embodiment of the invention comprises an ointment comprising one or more antifungals, one or more water insoluble (or lipophilic) components and one or more water soluble (or hydrophilic) components. The composition preferably has the consistency of an ointment for treatment of oral fungal infections or vaginal yeast or fungal infections, including but not limited to those caused by a fungus called Candida. The antifungal may be any antifungal, which is effective to treat oral fungal or vaginal yeast or fungal infections, including but not limited to immidazole derivatives, preferably miconazole nitrate, cyclopirox, clotrimazole, econazole, saperconazole, fenticonazole, sertaconazole, terconazole, itraconazole, ketoconazole, butaconazole, tioconazole, posaconazole, fluconazole, cyclopirox, their pharmaceutically acceptable salts or a combination thereof, and more preferably miconazole nitrate. The antifungal is present preferably in amounts from about 400 mg to about 1200 mg per dose.

Ointments in the prior art do not have bases comprised of water insoluble and water soluble components. The water insoluble components may be any water insoluble components, which are acceptable for application to and not unduly irritating to the body cavity, including but not limited to stearyl alcohol, petrolatum, vegetable oil suppository bases or a combination thereof, preferably stearyl alcohol. The water soluble components may be any water soluble components, which are acceptable for application to and not unduly irritating to the body cavity, including but not limited to polyethylene glycols, propylene glycols and glycerin.

Single dose treatments are most preferable, while multiple doses may also be included in the invention. Ointments used in such treatments should adhere to the vaginal mucous membrane, not leak or wash out from the vagina, and continue to release the antifungal, antibacterial or a both into the vagina for more than 24 hours, preferably for about 72 to about 120 hours, most preferably for at least 70 hours.

The present invention utilizes a combination of water soluble and water insoluble components, which promotes the retention of the antifungal and the effective allocation of antifungal to the water-soluble components. The combination of water soluble and water insoluble components are utilized for the base. The water soluble components preferably include components with a mixture of both high and low melting points, more preferably polyethylene glycols, propylene glycols and glycerin. The water soluble components are preferably about 15% to about 40% of total ointment. More preferably, the range should be from about 15% to about 35%. Most preferably, polyethylene glycol 400, which is a liquid, is combined with polyethylene glycol 3350, which is a solid in a weight ratio of 5:2. Preferably, the water insoluble component should be present in the composition in the amount of from about 30% to about 45% by weight of the composition. The combination has a melting range of about 35 to about 38° C. Additionally, the water insoluble components preferably include components with a mixture of both high and low melting points, more preferably petrolatum and vegetable oils with both high and low melting points. High melting points are in the range of about 38 to about 42° and low melting points are in the range of about 33 to about 37° C.

The ratio of water soluble to water insoluble components can be varied in order to place the antifungal in the water insoluble or water soluble component of the ointment. The preferred ratio of water soluble to water insoluble components is about 2:3 to about 3:4. Additionally, polysorbate 60 may be added to the water soluble components in order to increase the percent of antifungal in the water soluble component. Too little antifungal in the water soluble components will decrease the efficacy of the antifungal or antibacterial, while too much antifungal in the water soluble component will increase its toxicity to the consumer and potential for irritation. The infection is treated at the mucosa and therefore, it is important to retain most of the antifungal or antibacterial at the body cavity mucosa and maintain its concentration for a long period of time, preferably at least 24 hours, more preferably 72 hours and most preferably 120 hours. The preferred embodiment delivers an effective amount of the antifungal, which resides in the vagina for sufficient time after a single dose, to effectively treat the fungal infection without any additional doses (this is referred to as a single dose ointment).

Further, this embodiment may utilize bioadhesive agents which help to promote adhesion of the ointment to the body cavity mucosa membranes. The bioadhesive agents (including gelling agents and hydrocolloids) may be any bioadhesive agent which is acceptable for application to and not unduly irritating to the body cavity, preferably xanthan gum, sodium carboxymethylcellulose, or mixtures thereof, most preferably a mixture of xanthan gum and sodium carboxymethylcellulose. The fungal infection is located at the body cavity mucous membranes, and the longer residence time of the composition over the prior art promotes the effectiveness of the invention. The bioadhesive agents allow the ointment to be applied and melted in the vagina, where the ointment comes into contact with moisture. Then, the ointment gels and therefore the antifungal is retained for sufficient time to effectively treat the infection.

Additionally, this embodiment may include one or more dispersing agents, which may be any dispersing agents acceptable for application to and not unduly irritating to the body cavity, preferably silicon dioxide. Dispersing agents contribute homogenous melt and spread characteristics to the mixture and aids in the adhesion to the body cavity mucous membrane for a controlled release of the antifungal or antibacterial.

Additionally, this embodiment may include one or more antibacterials. The antibacterials may be any antibacterials which are effective to treat bacterial infections, are acceptable for application to and not unduly irritating to the body cavity. Preferably, the antibacterials are metronidazole, ornidazole, tinidazole, clindamycin, secnidazole, sodium polystyrene sulfate, sodium cellulose sulfate, or mixtures thereof, most preferably metronidazole.

Another embodiment of the invention comprises an ointment for vaginal use comprising one or more antifungals, one or more water insoluble components, one or more water soluble components, and one or more probiotics. The probiotic is preferably probiotic organisms, including but not limited to Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbweckii, L. helveticus, L. salivarius, L. collinoides, L. buchneri, L. rogosal, L. bifidum, B. bifidum, B. breve, B. adolescetis or B. longum.

Another embodiment of the invention comprises an ointment for vaginal use comprising one or more antibacterial, one or more water insoluble components, one or more water soluble components, and one or more probiotics. The probiotic is preferably probiotic organisms, including but not limited to Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbweckii, L. helveticus, L. salivarius, L. collinoides, L. buchneri, L. rogosal, L. bifidum, B. bifidum, B. breve, B. adolescetis or B. longum.

Another embodiment of the invention comprises an ointment comprising one or more antifungals, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Another embodiment of the invention comprises an ointment comprising one or more antibacterials, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Probiotics may be incorporated into the embodiment for the establishment and maintenance of the healthy vaginal flora.

Another embodiment of the invention comprises an ointment comprising one or more antifungals, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Another embodiment of the invention comprises an ointment comprising one or more antibacterials, one or more water insoluble components, one or more water soluble components and one or more antivirals. The antiviral may preferably include but is not limited to immunomodulators, more preferably imiquimod, its derivatives, podofilox, podophyllin, interferon alpha, reticulos, and cidofovir.

Antivirals may be incorporated into the embodiment for treatment of viral infections, including but not limited to genital human papillomavirus (HPV) infections, genital warts, herpes simplex infections and acquired immunodeficiency syndrome (AIDS).

Another embodiment of the invention comprises an ointment comprising one or more antibacterials, one or more water insoluble (or lipophilic) components and one or more water soluble (or hydrophilic) components. The ointment is used to treat body cavity bacterial infections, including but not limited to bacterial vaginosis. The antibacterial may be any antibacterial, which is effective to treat body cavity bacterial infections, including but not limited to metronidazole, secnidazole, sodium polystyrene sulfate, sodium cellulose sulfate or a combination thereof, and more preferably metronidazole. The antibacterial is present in amounts from about 25 mg to about 250 mg per dose.

The water insoluble components may be any water insoluble components, which are acceptable for application to and not unduly irritating to the body cavity, including but not limited to petrolatum, vegetable oil bases or a combination thereof. The water soluble components may be any water soluble components, which are acceptable for application to and not unduly irritating to the body cavity, including but not limited to polyethylene glycols, propylene glycols and glycerin.

The combination of water soluble and water insoluble components are utilized for the base. The water soluble components preferably include components with a mixture of both high and low melting points, more preferably polyethylene glycols, propylene glycols and glycerin. Most preferably, polyethylene glycol 400, which is a liquid, is combined with polyethylene 3350, which is a solid. Additionally, the water insoluble components preferably include components with a mixture of both high and low melting points, more preferably petrolatum and vegetable oils with both high and low melting points.

The ratio of water soluble to water insoluble components can be varied in order to place the antibacterial in the water insoluble or water soluble component of the ointment. The preferred ratio of water soluble to water insoluble components is 2:3. Additionally, polysorbate 60 may be added to the water soluble components in order to increase the percent of antibacterial in the water soluble component. Too little antibacterial in the water soluble components will decrease the efficacy of the antibacterial, while too much antibacterial in the water soluble component will increase its toxicity to the consumer and potential for irritation. Too much antibacterial in the water soluble component will also increase the antibacterial concentration in the blood and decrease its concentration at the body cavity mucosa. The infection is treated at the mucosa and therefore, it is important to retain most of the antibacterial at the body cavity mucosa and maintain its concentration for a long period of time, preferably at least 24 hours, more preferably 72 hours and most preferably 120 hours. The preferred embodiment delivers an effective amount of the antibacterial, which resides in the body cavity for sufficient time after a single dose, to effectively treat the bacterial infection with any additional doses (this is referred to as a single dose ointment).

Further, this embodiment may utilize bioadhesive agents which help to promote adhesion of the ointment to the vaginal mucosa membranes. The bioadhesive agents (including gelling agents and hydrocolloids) may be any bioadhesive agent which is acceptable for application to and not unduly irritating to the body cavity, preferably xanthan gum, sodium carboxymethylcellulose, or mixtures thereof, most preferably a mixture of xanthan gum and sodium carboxymethylcellulose. The bacterial infection is located at the body cavity mucosa membranes, and the longer residence time of the composition over the prior art promotes the effectiveness of the invention. Further, the bioadhesive agents retain the antibacterial in the body cavity mucosa membranes and prolongs antibacterial action. The bioadhesive agents allow the ointment to be applied and melted in the body cavity, where the ointment comes into contact with moisture. Then, the ointment gels and therefore the antibacterial is retained for sufficient time to effectively treat the infection.

Additionally, this embodiment may include one or more dispersing agents, which may be any dispersing agents, emulsifiers and non-emulsifiers, acceptable for application to and not unduly irritating to the body cavity, preferably silicon dioxide. Dispersing agents contribute homogenous melt and spread characteristics to the mixture and aids in the adhesion to the body cavity mucous membrane for a controlled release of the antifungal or antibacterial.

Another embodiment of the invention is the method of treating a fungal infection of a body cavity, preferably in a single dose. Ointments may be applied to the body cavity, and spread in the body cavity. At least part, and preferably all of, the ointment is melted, preferably on contact with the body and most preferably from body heat. The ointment is spread preferably substantially uniformly in the body cavity, preferably after the melting of the ointment has occurred. The ointment used may be, including but not limited to, any embodiment described above. The method may optionally also include treating a bacterial infection of a body cavity. The compositions and methods of this invention may, preferably, be applied to other mucosal membranes, including, but not limited to, the buccal mucosa and the nasal mucosa.Another embodiment of the invention is the method of treating a bacterial infection of a body cavity, preferably in a single dose. Ointments may be applied to the body cavity, and spread in the body cavity. At least part, and preferably all of, the ointment is melted, preferably on contact with the body and most preferably from body heat. The ointment is spread preferably substantially uniformly in the body cavity, preferably after the melting of the ointment has occurred. The ointment used may be, including but not limited to, any embodiment described above.

Another embodiment of the invention comprises the method of delivering an antifungal, antibacterial, antiviral or any combination thereof ointment (for example those described in the other embodiments) into the vaginal cavity with the use of a vaginal applicator. The vaginal applicator may be disposable, re-usable, or prefilled. Such vaginal applicators are known in the art and are used in connection with products such as Monistat® 1-Day vaginal ointment, Monistat® 3 Cream, and Monistat® 7 Cream (by McNeil—PPC, Inc., Johnson & Johnson, New Jersey).

Another embodiment of the invention comprises a method of delivering an antifungal, antibacterial, antiviral or any combination thereof ointment (for example those described in the other embodiments) into the vaginal cavity in a gelatin capsule with or without an applicator. Such gelatin capsules are known in the art and are used in connection with products such as Monistat® 1 combination pack (by McNeil—PPC, Inc., Johnson & Johnson, New Jersey). The gelatin capsule may comprise a soft gelatin capsule shell or a two piece hard gelatin capsule shell, preferably a soft gelatin capsule shell. The shell encloses the antifungal, antibacterial, antiviral or any combination thereof ointment (as claimed and taught herein).

The following examples are preferred embodiments of the invention. Examples 1 and 2 are the most preferred embodiments.

### EXAMPLE 1

| | |
|---|---|
| Miconazole Nitrate | 16.00% |
| White Petrolatum | 25.00% |
| Wecobee M (Wecobee is | 16.00% |
| a vegetable oil base.) | |
| Polyethylene Glycol 400 | 20.00% |
| Polyethylene Glycol 3350 | 8.00% |
| Stearyl Alcohol | 3.50% |
| Colloidal Silicon Dioxide | 1.50% |
| Sodium Carboxymethylcellulose | 7.00% |
| Xanthan Gum | 3.00% |

### EXAMPLE 2

| | |
|---|---|
| Miconazole Nitrate | 24.00% |
| White Petrolatum | 20.00% |
| Wecobee M | 13.00% |
| Polyethylene Glycol 400 | 20.00% |
| Polyethylene Glycol 3350 | 8.00% |
| Stearyl Alcohol | 3.50% |
| Colloidal Silicon Dioxide | 1.50% |
| Sodium Carboxymethylcellulose | 7.00% |
| Xanthan Gum | 3.00% |

### EXAMPLE 3

| | |
|---|---|
| Miconazole Nitrate | 16.00% |
| White petrolatum | 31.00% |
| Wecobee FS (Wecobee FS | 10.00% |
| has a higher melting point | |
| than Wecobee M.) | |
| Polyethylene Glycol 400 | 20.00% |
| Polyethylene Glycol 3350 | 8.00% |
| Stearyl Alcohol | 3.50% |
| Colloidal Silicon Dioxide | 1.50% |
| Sodium Carboxymethylcellulose | 7.00% |
| Xanthan Gum | 3.00% |

### EXAMPLE 4

| | |
|---|---|
| Miconazole Nitrate | 16.00% |
| White Petrolatum | 41.00% |
| Polyethylene Glycol 400 | 20.00% |
| Polyethylene Glycol 3350 | 8.00% |
| Stearyl Alcohol | 3.50% |
| Colloidal Silicon Dioxide | 1.50% |
| Sodium Carboxymethylcellulose | 7.00% |
| Xanthan Gum | 3.00% |

### EXAMPLE 5

| | |
|---|---|
| Miconazole Nitrate | 36.00% |
| Wecobee M | 10.00% |
| Wecobee FS | 44.00% |
| Colloidal Silicon Dioxide | 1.00% |
| Sodium Carboxymethylcellulose | 8.00% |
| Xanthan Gum | 1.00% |

### EXAMPLE 6

| | |
|---|---|
| Miconazole Nitrate | 8.00% |
| Polysorbate 60 | 3.00% |
| White Petrolatum | 38.00% |
| Polyethylene Glycol 400 | 26.00% |
| Polyethylene Glycol 3350 | 10.00% |
| Stearyl alcohol | 3.50% |
| Colloidal Silicon Dioxide | 1.50% |
| Sodium Carboxymethylcellulose | 7.00% |
| Xanthan Gum | 3.00% |

### EXAMPLE 7

| | |
|---|---|
| Miconazole Nitrate | 16.00% |
| Wecobee M | 26.00% |
| White Petrolatum | 55.40% |
| Polyethylene Oxide | 0.50% |
| Soy Lecithin | 0.50% |
| Colloidal Silicon Dioxide | 1.50% |
| Xanthan Gum | 3.00% |

### EXAMPLE 8

| | |
|---|---|
| Metronidazole | 0.75% |
| Polyethylene Glycol 400 | 60.00% |
| Polyethylene Glycol 3350 | 24.25% |
| Stearyl Alcohol | 3.50% |
| Colloidal Silicon Dioxide | 1.50% |
| Sodium Carboxymethylcellulose | 7.00% |
| Xanthan Gum | 3.00% |

### EXAMPLE 9

| | |
|---|---|
| Secnidazole | 1.00% |
| Polyethylene Glycol 400 | 60.00% |
| Polyethylene Glycol 3350 | 24.00% |
| Stearyl Alcohol | 3.50% |
| Colloidal Silicon Dioxide | 1.50% |
| Sodium Carboxymethylcellulose | 7.00% |
| Xanthan Gum | 3.00% |

### EXAMPLE 10

| | |
|---|---|
| Sodium Polystyrene Sulfonate | 5.00% |
| Polyethylene Glycol 400 | 55.00% |
| Polyethylene Glycol 3350 | 25.00% |
| Stearyl Alcohol | 3.50% |
| Colloidal Silicon Dioxide | 1.50% |
| Sodium Carboxymethylcellulose | 7.00% |
| Xanthan Gum | 3.00% |

### EXAMPLE 11

| | |
|---|---|
| Sodium Cellulose Sulfate | 6.00% |
| Polyethylene Glycol 400 | 55.00% |
| Polyethylene Glycol 3350 | 24.00% |
| Stearyl Alcohol | 3.50% |
| Colloidal Silicon Dioxide | 1.50% |
| Sodium Carboxymethylcellulose | 7.00% |
| Xanthan Gum | 3.00% |

The invention also includes a method of identifying a vaginal antifungal ointment, which resides in the vagina long enough to be effective after a single dose. An antifungal ointment is applied to the vagina of a mammal, preferably a mammal, and blood samples are taken at set time intervals. The samples are tested for the concentration of antifungal in the blood, and the data is recorded. It must be determined whether the data is above a minimum concentration for antifungal, preferably about 1.0 ηg/ml and below a maximum concentration below its toxicity level for at least as long a time to be effective to treat the infection, preferably over about 24 hours, most preferably from about 72 to about 120 hours. The ointment may be including but not limited to any embodiment described above.

The invention also includes a method of identifying a vaginal antibacterial ointment, which is effective after a single dose. An antibacterial ointment is applied to the vagina of a consumer, and blood samples are taken at set time intervals. The samples are tested for the concentration of antibacterial in the blood, and the data is recorded. Finally, it must be determined whether the data is above a minimum concentration of antibacterial, and below a maximum concentration for at least as long a time to be effective for treating the infection, preferably over about 24 hours, most preferably from about 72 to about 120 hours. The ointment may be including but not limited to any embodiment described above.

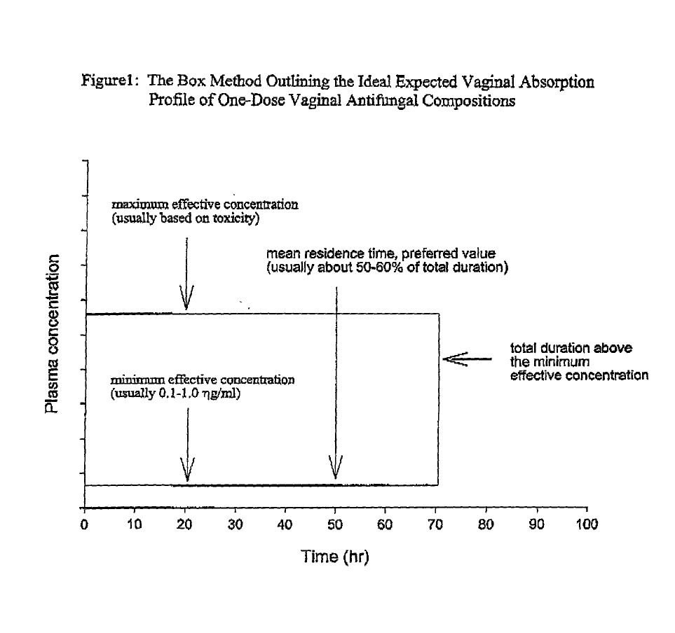

FIG. 1 is a graph showing the absorption profile of the ideal single dose vaginal antifungal ointment or antibacterial ointment. Absorption profile means the concentration of the antifungal (the y axis in FIG. 1) in the blood of a user of the antifungal ointment or antibacterial ointment over a set period of time (the x axis in FIG. 1). In FIG. 1, 1 denotes the minimum effective concentration, preferably about 0.1 1.0 ηg/ml. The maximum effective concentration of antifungal in the blood may be determined by toxicity and or irritation. When performing the method of identifying an effective single dose vaginal antifungal ointment or antibacterial ointment, data may be recorded on a graph such as FIG. 1 to aid in the determining of whether the data indicates an effective ointment.

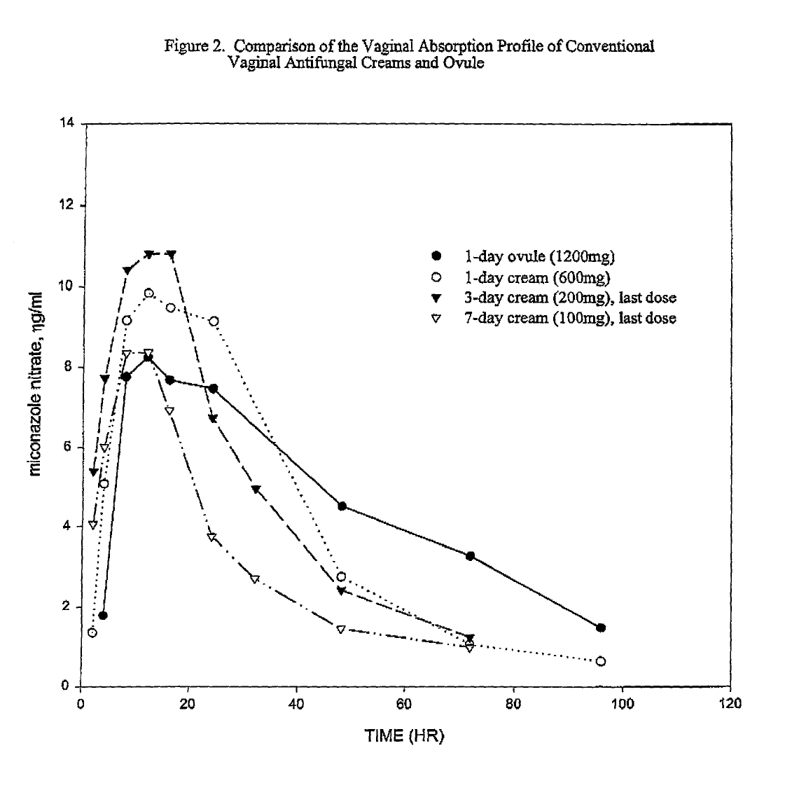

FIG. 2 is a graph showing the absorption profiles of Monistat® 1 Dual Pak, a 600 mg one-day cream, 200 mg three-day cream, and 100 mg seven-day cream. Monistat® 1 Dual Pak is a single dose 1200 mg soft gelatin ovule in the prior art. However, the data shows that the one-day, three-day and seven-day creams do not maintain antifungal concentrations of higher than 2 ηg/ml beyond 50 hours, while the antifungal concentration for Monistat® 1 Dual Pak 1200 mg soft gelatin ovule was higher than 2 ηg/ml even after 100 hours.

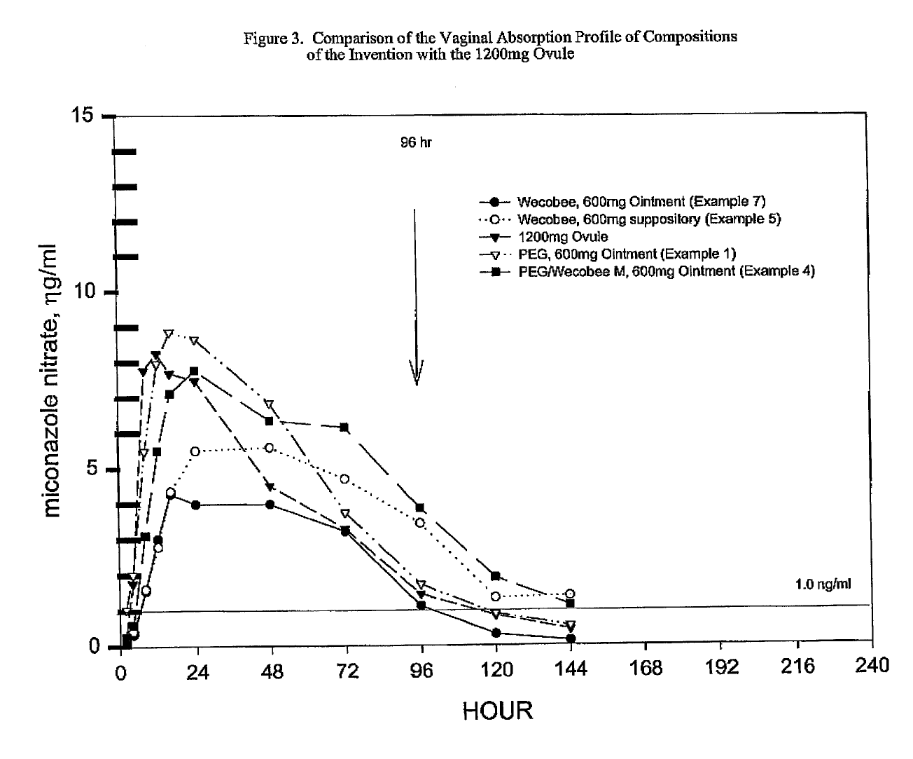

FIG. 3 is a graph showing the absorption profiles of Monistat® 1 Dual Pak 1200 mg soft gelatin ovule with four embodiments of the invention.

The ointments of Examples 1, 4, 5, and 7 were tested and compared with Monistat® 1 Dual Pak 1200 mg soft gelatin ovule. Blood samples were drawn from users of the ointments at 2, 4, 8, 12, 16, 24, 48, 72, 96, 120 and 144 hours after application of the ointment. All of the ointments had a sufficient concentration of the antifungal for a sufficient time (as displayed in FIG. 3) to treat a fungal vaginal infection with a single dose. Additionally, Monistat® had been clinically tested in the prior art to determine whether a single dose was effective to treat fungal vaginal infections. Without doing clinical efficacy tests on Examples 1, 4, 5, and 7, through the comparison of blood tests to a known single dose composition, these Examples are found to be efficacious after a single dose. These examples delivered effective antifungal even though each contained about half (approximately 600 mg/dose) as much antifungal as Monistat® 1 (approximately 1200 mg/dose). The method for determining the residence time in the vagina of an antifungal ointment may be used as described in this paragraph rather than the extensive clinical testing used in the prior art. Monistat's® (a clinically proven prior art single dose compositions) data in FIG. 3 was comparable to the embodiments of the invention tested.

It is understood that while the invention has been described in conjunction with the detailed description thereof, that the foregoing description is intended to illustrate and not limit the scope of the invention, which is defined by the scope of the appended claims. Other aspects, advantages, and modifications are evident from a review of the following claims. What is claimed is:

1. An antifungal ointment comprising
One or more antifungals;
One or more water insoluble components; and
One or more water soluble components:

2. The ointment of claim 1 wherein the water insoluble components comprise one or more of the group consisting of petrolatum and vegetable oil base.

3. The ointment of claim 1 wherein the water insoluble components comprise stearyl alcohol.

4. The ointment of claim 1 wherein the water insoluble components have a mixture of high and low melting points.

5. The ointment of claim 1 wherein the water soluble components comprise one or more components selected from the group consisting of polyethylene glycols, propylene glycols and glycerin.

6. The ointment of claim 1 wherein the water soluble components comprise one or more polyethylene glycols.

7. The ointment of claim 1 wherein the water soluble and water insoluble components are present in a ratio of from about 2:3 to about 3:4.

8. The ointment of claim 1 wherein the water soluble and water insoluble components are in a ratio, wherein the ratio causes the antifungal to be at least partially present in the water soluble component.

9. The ointment of claim 1 further comprising one or more nonionic surfactants.

10. The ointment of claim 9 wherein the surfactant comprises polysorbate 60.

11. The ointment of claim 1 further comprising one or more bioadhesive agents.

12. The ointment of claim 11 wherein the bioadhesive agents comprise one or more of the group consisting of xanthan gum and sodium carboxymethylcellulose.

13. The ointment of claim 11 wherein the bioadhesive agents comprise xanthan gum and sodium carboxymethylcellulose.

14. The ointment of claim 11 wherein the bioadhesive agents promote adhesion of the ointment to vaginal mucosa membranes.

15. The ointment of claim 11 wherein the bioadhesive agents retain the antifungal in vaginal mucosa membranes and prolongs antifungal action.

16. The ointment of claim 1 further comprising one or more dispersing agents.

17. The ointment of claim 16 wherein the dispersing agents comprise silicon dioxide.

18. The ointment of claim 1 wherein the antifungal comprises one or more of the group consisting of miconazole nitrate, cyclopirox, clotrimazole, econazole, saperconazole, terconazole, fenticonazole, sertaconazole, posaconazole, itraconazole, ketoconazole, butaconazole, tioconazole, fluconazole, and their pharmaceutically acceptable salts.

19. The ointment of claim 1 wherein the antifungal is miconazole nitrate.

20. The ointment of claim 1 wherein the antifungal is present in an amount from about 400 mg to about 1200 mg.

21. The ointment of claim 1 wherein the antifungal is effective in a single dose.

22. The ointment of claim 1 further comprising one or more antibacterials.

23. The ointment of claim 22 wherein the antibacterial comprises one or more of the group consisting of metronidazole, secnidazole, ornidazole, tinidazole, clindamycin, sodium polystyrene sulfate, and sodium cellulose sulfate.

24. The ointment of claim 22 wherein the antibacterial comprises metronidazole.

25. The ointment of claim 1 further comprising one or more probiotics.

26. The ointment of claim 25 wherein the probiotics comprise one or more of the group consisting of organisms of the species Lactobacillus and Bifidobacterium.

27. The ointment of claim 25 wherein the probiotics comprise one or more of the group consisting of L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis and B. longum.

28. The ointment of claim 1 further comprising one or more antivirals.

29. The ointment of claim 1 wherein the antivirals comprise immunomodulators.

30. The ointment of claim 1 wherein the antivirals comprise one or more of the group consisting of imiquimod, imiquimod derivatives, podofilox, podophyllin, interferon alpha, reticulos, and cidofovir.

31. An antibacterial ointment comprising
One or more antibacterial;
One or more water insoluble components; and
One or more water soluble components.

32. The ointment of claim 31 wherein the water insoluble components comprise one or more of the group consisting of petrolatum and vegetable oil base.

33. The ointment of claim 31 wherein the water insoluble components comprise stearyl alcohol.

34. The ointment of claim 31 wherein the water insoluble components have a mixture of high and low melting points.

35. The ointment of claim 31 wherein the water soluble components comprise one or more components selected from the group consisting of polyethylene glycols, propylene glycols and glycerin.

36. The ointment of claim 31 wherein the water soluble components comprise one or more polyethylene glycols.

37. The ointment of claim 31 wherein the water soluble and water insoluble components are present in a ratio of from about 2:3 to about 3:4.

38. The ointment of claim 31 wherein the water soluble and water insoluble components are in a ratio, wherein the ratio causes the antibacterial to be at least partially present in the water soluble component.

39. The ointment of claim 31 further comprising one or more nonionic surfactants.

40. The ointment of claim 39 wherein the surfactant comprises polysorbate 60.

41. The ointment of claim 31 further comprising one or more bioadhesive agents.

42. The ointment of claim 41wherein the bioadhesive agents comprise one or more of the group consisting of xanthan gum and sodium carboxymethylcellulose.

43. The ointment of claim 41 wherein the bioadhesive agents comprise xanthan gum and sodium carboxymethylcellulose.

44. The ointment of claim 41 wherein the bioadhesive agents promote adhesion of the ointment to vaginal mucosa membranes.

45. The ointment of claim 41 wherein the bioadhesive agents retain the antibacterial in vaginal mucosa membranes and prolongs antibacterial action.

46. The ointment of claim 31 further comprising one or more dispersing agents.

47. The ointment of claim 46 wherein the dispersing agents comprise silicon dioxide.

48. The ointment of claim 31 wherein the antibacterial comprises one or more of the group consisting of metronidazole, secnidazole, ornidazole, tinidazole, clindamycin, sodium polystyrene sulfate, and sodium cellulose sulfate.

49. The ointment of claim 31 wherein the antibacterial comprises metronidazole.

50. The ointment of claim 31 wherein the antibacterial is effective in a single dose.

51. The ointment of claim 31 further comprising one or more probiotics.

52. The ointment of claim 51 wherein the probiotics comprise one or more of the group consisting of organisms of the species Lactobacillus and Bifidobacterium.

53. The ointment of claim 51 wherein the probiotics comprise one or more of the group consisting of L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis and B. longum.

54. The ointment of claim 31 further comprising one or more antivirals.

55. The ointment of claim 54 wherein the antivirals comprise immunomodulators.

56. The ointment of claim 54 wherein the antivirals comprise one or more of the group consisting of imiquimod, imiquimod derivatives, podofilox, podophyllin, interferon alpha, reticulos, and cidofovir.

57. An antifungal ointment comprising
Miconazole nitrate;
White petrolatum;
Vegetable oil base;
Polyethylene glycol 400;
Polyethylene glycol 3350;
Stearyl alcohol;
Colloidal silicon dioxide;
Sodium carboxymethylcellulose; and
Xanthan gum.

58. An antifungal ointment comprising
Miconazole nitrate;
Vegetable oil base;
Colloidal Silicon Dioxide;
Sodium Carboxymethylcellulose; and
Xanthan Gum.

59. An antifungal ointment comprising
Miconazole nitrate;
Polysorbate 60;
White petrolatum;
Polyethylene glycol 400;
Polyethylene glycol 3350;
Stearyl alcohol;
Colloidal silicon dioxide;
Sodium carboxymethylcellulose; and
Xanthan gum.

60. An antifungal ointment comprising
Miconazole nitrate;
Vegetable oil base;
White petrolatum;
Polyethylene oxide;
Soy lecithin;
Colloidal silicon dioxide; and
Xanthan gum.

61. An antibacterial ointment comprising
Metronidazole;
Polyethylene glycol 400;
Polyethylene glycol 3350;
Stearyl alcohol;
Colloidal silicon dioxide;
Sodium carboxymethylcellulose; and
Xanthan gum.

62. An antibacterial ointment comprising
Secnidazole;
Polyethylene glycol 400;
Polyethylene glycol 3350;
Stearyl alcohol;
Colloidal silicon dioxide;
Sodium carboxymethylcellulose; and
Xanthan gum.

63. An antibacterial ointment comprising
Sodium polystyrene sulfonate;
Polyethylene glycol 400;
Polyethylene glycol 3350;
Stearyl alcohol;
Colloidal silicon dioxide;
Sodium carboxymethylcellulose; and
Xanthan gum.

64. An antibacterial ointment comprising
Sodium cellulose sulfate;
Polyethylene glycol 400;
Polyethylene glycol 3350;
Stearyl alcohol;
Colloidal silicon dioxide;
Sodium carboxymethylcellulose; and
Xanthan gum.

65. An antiviral ointment comprising
One or more antiviral;
One or more water insoluble components; and
One or more water soluble components.

66. The ointment of claim 65 wherein the water insoluble components comprise one or more of the group consisting of petrolatum and vegetable oil base.

67. The ointment of claim 65 wherein the water insoluble components comprise stearyl alcohol.

68. The ointment of claim 65 wherein the water insoluble components have a mixture of high and low melting points.

69. The ointment of claim 65 wherein the water soluble components comprise one or more components selected from the group consisting of polyethylene glycols, propylene glycols and glycerin.

70. The ointment of claim 65 wherein the water soluble components comprise one or more polyethylene glycols.

71. The ointment of claim 65 wherein the water soluble and water insoluble components are present in a ratio of from about 2:3 to about 3:4.

72. The ointment of claim 65 wherein the water soluble and water insoluble components are in a ratio, wherein the ratio causes the antiviral to be at least partially present in the water soluble component.

73. The ointment of claim 65 further comprising one or more nonionic surfactants.

74. The ointment of claim 73 wherein the surfactant comprises polysorbate 60.

75. The ointment of claim 65 further comprising one or more bioadhesive agents.

76. The ointment of claim 75 wherein the bioadhesive agents comprise one or more of the group consisting of xanthan gum and sodium carboxymethylcellulose.

77. The ointment of claim 75 wherein the bioadhesive agents comprise xanthan gum and sodium carboxymethylcellulose.

78. The ointment of claim 75 wherein the bioadhesive agents promote adhesion of the ointment to vaginal mucosa membranes.

79. The ointment of claim 75 wherein the bioadhesive agents retain the antibacterial in vaginal mucosa membranes and prolongs antibacterial action.

80. The ointment of claim 65 further comprising one or more dispersing agents.

81. The ointment of claim 80 wherein the dispersing agents comprise silicon dioxide.

82. The ointment of claim 65 further comprising one or more probiotics.

83. The ointment of claim 82 wherein the probiotics comprise one or more of the group consisting of organisms of the species Lactobacillus and Bifidobacterium.

84. The ointment of claim 82 wherein the probiotics comprise one or more of the group consisting of L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis and B. longum.

85. The ointment of claim 65 wherein the antivirals comprise immunomodulators.

86. The ointment of claim 65 wherein the antivirals comprise one or more of the group consisting of imiquimod, imiquimod derivatives, podofilox, podophyllin, interferon alpha, reticulos, and cidofovir.

87. The method of treating a fungal infection of a body cavity comprising
Applying an ointment to the body cavity; wherein the ointment comprises an antifungal, one or more water soluble components and one or more water insoluble components;
Spreading the ointment substantially uniformly in the body cavity;
Melting at least a part of the ointment; and
Retaining the ointment in the body cavity.

88. The method of claim 87 wherein the applying occurs only once.

89. The method of claim 87 wherein the body cavity is a vagina.

90. The method of claim 87 wherein the body cavity is an oral cavity.

91. The method of claim 87 wherein the ointment has a melting point at about body temperature.

92. The method of claim 87 wherein the water insoluble components comprise one or more of the group consisting of petrolatum and vegetable oil base.

93. The method of claim 87 wherein the water insoluble components comprise stearyl alcohol.

94. The method of claim 87 wherein the water insoluble components have a mixture of high and low melting points.

95. The method of claim 87 wherein the water soluble components comprise one or more components selected from the group consisting of polyethylene glycols, propylene glycols and glycerin.

96. The ointment of claim 87 wherein the water soluble components comprise one or more polyethylene glycols.

97. The method of claim 87 wherein the water soluble and water insoluble components are present in a ratio of from about 2:3 to about 3:4.

98. The method of claim 87 wherein the water soluble and water insoluble components are in a ratio, wherein the ratio causes the antifungal to be at least partially present in the water soluble component.

99. The method of claim 87 further comprising one or more nonionic surfactants.

100. The method of claim 99 wherein the surfactant comprises polysorbate 60.

101. The method of claim 87 further comprising one or more bioadhesive agents.

102. The method of claim 101 wherein the bioadhesive agents comprise one or more of the group consisting of xanthan gum and sodium carboxymethylcellulose.

103. The method of claim 101 wherein the bioadhesive agents comprise xanthan gum and sodium carboxymethylcellulose.

104. The method of claim 101 wherein the bioadhesive agents promote adhesion of the ointment to vaginal mucosa membranes.

105. The method of claim 101 wherein the bioadhesive agents retain the antifungal in vaginal mucosa membranes and prolong antifungal action.

106. The method of claim 87 further comprising one or more dispersing agents.

107. The method of claim 106 wherein the dispersing agents comprise silicon dioxide.

108. The method of claim 87 wherein the antifungal comprises one or more of the group consisting of miconazole nitrate, cyclopirox, clotrimazole, econazole, saperconazole, terconazole, fenticonazole, sertaconazole, posaconazole, itraconazole, ketoconazole, butaconazole, tioconazole, fluconazole, and their pharmaceutically acceptable salts.

109. The method of claim 87 wherein the antifungal is miconazole nitrate.

110. The method of claim 87 wherein the antifungal is present in an amount from about 400 mg to about 1200 mg.

111. The method of claim 87 wherein the ointment further comprises an antibacterial.

112. The method of claim 87 wherein the ointment further comprises one or more probiotics.

113. The method of claim 112 wherein the probiotics comprise one or more of the group consisting of organisms of the species Lactobacillus and Bifidobacterium.

114. The ointment of claim 112 wherein the probiotics comprise one or more of the group consisting of L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis and B. longum.

115. The ointment of claim 87 wherein the ointment further comprises one or more antivirals.

116. The ointment of claim 115 wherein the antivirals comprise immunomodulators.

117. The ointment of claim 115 wherein the antivirals comprise one or more of the group consisting of imiquimod, imiquimod derivatives, podofilox, podophyllin, interferon alpha, reticulos, and cidofovir.

118. The method of treating a bacterial infection of a body cavity comprising
Applying an ointment to the body cavity; wherein the ointment comprises one or more antibacterials, one or more water soluble components, and one or more water insoluble components;
Spreading the ointment substantially uniformly in the body cavity;
Melting at least a part of the ointment; and
Retaining the ointment in the body cavity.

119. The method of claim 118 wherein the applying occurs only once.

120. The method of claim 118 wherein the body cavity is a vagina.

121. The method of claim 118 wherein the body cavity is an oral cavity.

122. The method of claim 118 wherein the water insoluble components comprise one or more of the group consisting of petrolatum and vegetable oil base.

123. The method of claim 118 wherein the ointment has a melting point at about body temperature.

124. The method of claim 118 wherein the water insoluble components comprise stearyl alcohol.

125. The method of claim 118 wherein the water insoluble components have a mixture of high and low melting points.

126. The method of claim 118 wherein the water soluble components comprise one or more components selected from the group consisting of polyethylene glycols, propylene glycols and glycerin.

127. The ointment of claim 118 wherein the water soluble components comprise one or more polyethylene glycols.

128. The method of claim 118 wherein the water soluble and water insoluble components are present in a ratio of from about 2:3 to about 3:4.

129. The method of claim 118 wherein the water soluble and water insoluble components are in a ratio, wherein the ratio causes the antibacterial to be at least partially present in the water soluble component.

130. The method of claim 118 wherein the ointment further comprises one or more nonionic surfactants.

131. The method of claim 130 wherein the surfactant comprises polysorbate 60.

132. The method of claim 118 wherein the ointment further comprises one or more bioadhesive agents.

133. The method of claim 132 wherein the bioadhesive agents comprise one or more of the group consisting of xanthan gum and sodium carboxymethylcellulose.

134. The method of claim 132 wherein the bioadhesive agents comprise xanthan gum and sodium carboxymethylcellulose.

135. The method of claim 132 wherein the bioadhesive agents promote adhesion of the ointment to vaginal mucosa membranes.

136. The method of claim 132 wherein the bioadhesive agents retain the antifungal in vaginal mucosa membranes and prolong antifungal action.

137. The method of claim 118 wherein the ointment further comprises one or more dispersing agents.

138. The method of claim 137 wherein the dispersing agents comprise silicon dioxide.

139. The method of claim 118 wherein the antibacterial comprises one or more of the group consisting of metronidazole, secnidazole, ornidazole, tinidazole, clindamycin sodium polystyrene sulfate, and sodium cellulose sulfate.

140. The method of claim 118 wherein the antibacterial is metronidazole.

141. The method of claim 118 wherein the ointment further comprises one or more probiotics.

142. The method of claim 141 wherein the probiotics comprise one or more of the group consisting of organisms of the species Lactobacillus and Bifidobacterium.

143. The method of claim 141 wherein the probiotics comprise one or more of the group consisting of L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis and B. longum.

144. The method of claim 118 wherein the ointment further comprises one or more antivirals.

145. The method of claim 144 wherein the antivirals comprise immunomodulators.

146. The method of claim 144 wherein the antivirals comprise one or more of the group consisting of imiquimod, imiquimod derivatives, podofilox, podophyllin, interferon alpha, reticulos, and cidofovir.

147. The method of treating a viral infection of a body cavity comprising
Applying an ointment to the body cavity; wherein the ointment comprises one or more antivirals, one or more water soluble components, and one or more water insoluble components;
Spreading the ointment substantially uniformly in the body cavity;
Melting at least a part of the ointment; and
Retaining the ointment in the body cavity.

148. The method of claim 147 wherein the applying occurs only once.

149. The method of claim 147 wherein the body cavity is a vagina.

150. The method of claim 147 wherein the body cavity is an oral cavity.

151. The method of claim 147 wherein the water insoluble components comprise one or more of the group consisting of petrolatum and vegetable oil base.

152. The method of claim 147 wherein the ointment has a melting point at about body temperature.

153. The method of claim 147 wherein the water insoluble components comprise stearyl alcohol.

154. The method of claim 147 wherein the water insoluble components have a mixture of high and low melting points.

155. The method of claim 147 wherein the water soluble components comprise one or more components selected from the group consisting of polyethylene glycols, propylene glycols and glycerin.

156. The ointment of claim 147 wherein the water soluble components comprise one or more polyethylene glycols.

157. The method of claim 147 wherein the water soluble and water insoluble components are present in a ratio of from about 2:3 to about 3:4.

158. The method of claim 147 wherein the water soluble and water insoluble components are in a ratio, wherein the ratio causes the antibacterial to be at least partially present in the water soluble component.

159. The method of claim 147 wherein the ointment further comprises one or more nonionic surfactants.

160. The method of claim 159 wherein the surfactant comprises polysorbate 60.

161. The method of claim 147 wherein the ointment further comprises one or more bioadhesive agents.

162. The method of claim 161 wherein the bioadhesive agents comprise one or more of the group consisting of xanthan gum and sodium carboxymethylcellulose.

163. The method of claim 161 wherein the bioadhesive agents comprise xanthan gum and sodium carboxymethylcellulose.

164. The method of claim 161 wherein the bioadhesive agents promote adhesion of the ointment to vaginal mucosa membranes.

165. The method of claim 161 wherein the bioadhesive agents retain the antifungal in vaginal mucosa membranes and prolong antifungal action.

166. The method of claim 147 wherein the ointment further comprises one or more dispersing agents.

167. The method of claim 166 wherein the dispersing agents comprise silicon dioxide.

168. The method of claim 147 wherein the antiviral comprises immunomodulators.

169. The method of claim 147 wherein the antiviral comprise one or more of the group consisting of imiquimod, imiquimod derivatives, podofilox, podophyllin, interferon alpha, reticulos, and cidofovir.

170. The method of claim 147 wherein the ointment further comprises one or more probiotics.

171. The method of claim 170 wherein the probiotics comprise one or more of the group consisting of organisms of the species Lactobacillus and Bifidobacterium.

172. The method of claim 170 wherein the probiotics comprise one or more of the group consisting of L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis and B. longum.

173. An antifungal ointment comprising
One or more antifungals;
One or more water insoluble components;
One or more water soluble components; and
One or more bioadhesive agents.

174. The ointment of claim 173 further comprising one or more dispersing agents.

175. The ointment of claim 173 wherein the water insoluble components comprise one or more of the group consisting of petrolatum and vegetable oil base.

176. The ointment of claim 173 wherein the water insoluble components comprise stearyl alcohol.

177. The ointment of claim 173 wherein the water insoluble components have a mixture of high and low melting points.

178. The ointment of claim 173 wherein the water soluble components comprise one or more components selected from the group consisting of polyethylene glycols, propylene glycols and glycerin.

179. The ointment of claim 173 wherein the water soluble components comprise one or more polyethylene glycols.

180. The ointment of claim 173 wherein the water soluble and water insoluble components are present in a ratio of from about 2:3 to about 3:4.

181. The ointment of claim 173 wherein the water soluble and water insoluble components are in a ratio, wherein the ratio causes the antifungal to be at least partially present in the water soluble component.

182. The ointment of claim 173 further comprising one or more nonionic surfactants.

183. The ointment of claim 182 wherein the surfactant comprises polysorbate 60.

184. The ointment of claim 173 wherein the bioadhesive agents comprise one or more of the group consisting of xanthan gum and sodium carboxymethylcellulose.

185. The ointment of claim 173 wherein the bioadhesive agents comprise xanthan gum and sodium carboxymethylcellulose.

186. The ointment of claim 173 wherein the bioadhesive agents promote adhesion of the ointment to vaginal mucosa membranes.

187. The ointment of claim 173 wherein the bioadhesive agents retain the antibacterial in vaginal mucosa membranes and prolongs antibacterial action.The ointment of claim 174 wherein the dispersing agents comprise silicon dioxide.

188. The ointment of claim 173 wherein the antifungal comprises one or more of the group consisting of miconazole nitrate, cyclopirox, clotrimazole, econazole, saperconazole, terconazole, fenticonazole, sertaconazole,posaconazole, itraconazole, ketoconazole, butaconazole, tioconazole, fluconazole, and their pharmaceutically acceptable salts.

189. The ointment of claim 173 wherein the antifungal is miconazole nitrate.

190. The ointment of claim 173 wherein the antifungal is present in an amount from about 400 mg to about 1200 mg.

191. The ointment of claim 173 wherein the antifungal is effective in a single dose.

192. The ointment of claim 173 further comprising an antibacterial.

193. The ointment of claim 192 wherein the antibacterial comprises one or more of the group consisting of metronidazole, secnidazole, ornidazole, tinidazole, clindamycin sodium polystyrene sulfate, and sodium cellulose sulfate.

194. The ointment of claim 192 wherein the antibacterial comprises metronidazole.

195. The ointment of claim 173 further comprising one or more probiotics.

196. The ointment of claim 195 wherein the probiotics comprise one or more of the group consisting of organisms of the species Lactobacillus and Bifidobacterium.

197. The ointment of claim 195 wherein the probiotics comprise one or more of the group consisting of L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis and B. longum.

198. The ointment of claim 173 further comprising one or more antivirals.

199. The ointment of claim 198 wherein the antivirals comprise immunomodulators.

200. The ointment of claim 198 wherein the antivirals comprise one or more of the group consisting of imiquimod, imiquimod derivatives, podofilox, podophyllin, interferon alpha, reticulos, and cidofovir.

201. An antibacterial ointment comprising
One or more antibacterial; and
One or more water insoluble components;
One or more water soluble components; and
One or more bioadhesive agents.

202. The ointment of claim 201 wherein the water insoluble components comprise one or more of the group consisting of petrolatum and vegetable oil base.

203. The ointment of claim 201 wherein the water insoluble components comprise stearyl alcohol.

204. The ointment of claim 201 wherein the water insoluble components have a mixture of high and low melting points.

205. The ointment of claim 201 wherein the water soluble components comprise one or more components selected from the group consisting of polyethylene glycols, propylene glycols and glycerin.

206. The ointment of claim 201 wherein the water soluble components comprise one or more polyethylene glycols.

207. The ointment of claim 201 wherein the water soluble and water insoluble components are present in a ratio of from about 2:3 to about 3:4.

208. The ointment of claim 201 wherein the water soluble and water insoluble components are in a ratio, wherein the ratio causes the antibacterial to be at least partially present in the water soluble component.

209. The ointment of claim 201 further comprising one or more nonionic surfactants.

210. The ointment of claim 209 wherein the surfactants comprise polysorbate 60.

211. The ointment of claim 201 wherein the antibacterial is in the water soluble component.

212. The ointment of claim 201 wherein the bioadhesive agents comprise one or more of the group consisting of xanthan gum and sodium carboxymethylcellulose.

213. The ointment of claim 201 wherein the bioadhesive agents comprise xanthan gum and sodium carboxymethylcellulose.

214. The ointment of claim 201 wherein the bioadhesive agents promote adhesion of the ointment to vaginal mucosa membranes.

215. The ointment of claim 201 wherein the bioadhesive agents retain the antibacterial in vaginal mucosa membranes and prolongs antibacterial action.

216. The ointment of claim 201 further comprising one or more dispersing agents.

217. The ointment of claim 216 wherein the dispersing agents comprise silicon dioxide.

218. The ointment of claim 201 wherein the antibacterial comprises one or more of the group consisting of metronidazole, secnidazole, ornidazole, tinidazole, clindamycin sodium polystyrene sulfate, and sodium cellulose sulfate.

219. The ointment of claim 201 wherein the antibacterial comprises metronidazole.

220. The ointment of claim 201 wherein the antibacterial is effective in a single dose.

221. The ointment of claim 201 further comprising one or more probiotics.

222. The ointment of claim 221 wherein the probiotics comprise one or more of the group consisting of organisms of the species Lactobacillus and Bifidobacterium.

223. The ointment of claim 221 wherein the probiotics comprise one or more of the group consisting of L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis and B. longum.

224. The ointment of claim 201 further comprising one or more antivirals.

225. The ointment of claim 224 wherein the antivirals comprise immunomodulators.

226. The ointment of claim 224 wherein the antivirals comprise one or more of the group consisting of imiquimod, imiquimod derivatives, podofilox, podophyllin, interferon alpha, reticulos, and cidofovir.

227. An antiviral ointment comprising
One or more antiviral;
One or more water insoluble components;
One or more water soluble components; and
One or more antiviral agents.

228. The ointment of claim 227 wherein the water insoluble components comprise one or more of the group consisting of petrolatum and vegetable oil base.

229. The ointment of claim 227 wherein the water insoluble components comprise stearyl alcohol.

230. The ointment of claim 227 wherein the water insoluble components have a mixture of high and low melting points.

231. The ointment of claim 227 wherein the water soluble components comprise one or more components selected from the group consisting of polyethylene glycols, propylene glycols and glycerin.

232. The ointment of claim 227 wherein the water soluble components comprise one or more polyethylene glycols.

233. The ointment of claim 227 wherein the water soluble and water insoluble components are present in a ratio of from about 2:3 to about 3:4.

234. The ointment of claim 227 wherein the water soluble and water insoluble components are in a ratio, wherein the ratio causes the antiviral to be at least partially present in the water soluble component.

235. The ointment of claim 227 further comprising one or more nonionic surfactants.

236. The ointment of claim 235 wherein the surfactant comprises polysorbate 60.

237. The ointment of claim 227 wherein the bioadhesive agents comprise one or more of the group consisting of xanthan gum and sodium carboxymethylcellulose.

238. The ointment of claim 227 wherein the bioadhesive agents comprise xanthan gum and sodium carboxymethylcellulose.

239. The ointment of claim 227 wherein the bioadhesive agents promote adhesion of the ointment to vaginal mucosa membranes.

240. The ointment of claim 227 wherein the bioadhesive agents retain the antibacterial in vaginal mucosa membranes and prolongs antibacterial action.

241. The ointment of claim 227 further comprising one or more dispersing agents.

242. The ointment of claim 241 wherein the dispersing agents comprise silicon dioxide.

243. The ointment of claim 227 further comprising one or more probiotics.

244. The ointment of claim 243 wherein the probiotics comprise one or more of the group consisting of organisms of the species Lactobacillus and Bifidobacterium.

245. The ointment of claim 243 wherein the probiotics comprise one or more of the group consisting of L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis and B. longum.

246. The ointment of claim 227 wherein the antivirals comprise immunomodulators.

247. The ointment of claim 227 wherein the antivirals comprise one or more of the group consisting of imiquimod, imiquimod derivatives, podofilox, podophyllin, interferon alpha, reticulos, and cidofovir.

248. A method of identifying a vaginal antifungal ointment suitable for use in a single dose application comprising
Applying an antifungal ointment to a vagina of a mammal;
Taking blood samples from the mammal at set time intervals;
Testing the samples for concentration of antifungal;
Recording data from the testing; and
Determining whether the data is above a minimum concentration for at least an effective time.

249. The method of claim 248 wherein the minimum concentration is 1.0 ηg/ml.

250. A method of identifying a vaginal antibacterial ointment suitable for use in a single dose application comprising
Applying an antibacterial ointment to a vagina of a mammal;
Taking blood samples from the mammal at set time intervals;
Testing the samples for concentration of antibacterial;
Recording data from the testing; and
Determining whether the data is above a minimum concentration for at least an effective time.

251. The method of claim 250 wherein the minimum concentration is 1.0 ηg/ml.

252. A method of identifying a vaginal antiviral ointment suitable for use in a single dose application comprising
Applying an antiviral ointment to a vagina of a mammal;
Taking blood samples from the mammal at set time intervals;
Testing the samples for concentration of antiviral;
Recording data from the testing; and
Determining whether the data is above a minimum concentration for at least an effective time.

253. The method of claim 252 wherein the minimum concentration is 1.0 ηg/ml
